(19) United States
(12) Patent Application Publication
(10) Pub. No.: US 2024/0292851 A1
(43) Pub. Date: Sep. 5, 2024

(54) NOVEL STRAINS OF CARNOBACTERIUM MALTAROMATICUM AND USES THEREOF

(71) Applicant: UNIVERSITE DE LORRAINE, NANCY (FR)

(72) Inventors: Frédéric BORGES, CHAVIGNY (FR); Anne-Marie REVOL-JUNELLES, VILLERS LES NANCY (FR)

(73) Assignee: UNIVERSITE DE LORRAINE, NANCY (FR)

(21) Appl. No.: 17/768,369

(22) PCT Filed: Oct. 14, 2020
(86) PCT No.: PCT/EP2020/078966

§ 371 Date: Apr. 12, 2022

(30) Foreign Application Priority Data
Oct. 24, 2019 (FR) FR1911895

(51) Int. Cl. A23C 9/12; A23C 3/08; A23C 19/032; A23C 19/10; C12N 1/20; C12R 1/01

(52) U.S. Cl. CPC A23C 9/1203; A23C 3/08; A23C 19/032; A23C 19/10; C12N 1/205; C12R 2001/01

(57) ABSTRACT

Strains of Carnobacterium maltaromaticum, a bacterial preparation including at least one strain of Carnobacterium maltaromaticum, and their use in the preparation of a food product. Also, a method for reducing microbial contamination during a process for the production of a fermented milk product to which a quantity of Carnobacterium maltaromaticum or a bacterial preparation including at least one strain of Carnobacterium maltaromaticum is added.

### DESCRIPTION

The present application relates to strains of Carnobacterium maltaromaticum, a bacterial preparation comprising at least one strain of Carnobacterium maltaromaticum, and their use in the preparation of a food product.

The presence of pathogenic bacteria in foodstuffs is a major public health problem. In the cheesemaking sector, the potential presence of Listeria monocytogenes is an ongoing concern. Its presence in transformed milk products has given rise to incidents which impact consumer health and the economic stability of the stakeholders in the sector. An example that can be mentioned is the heavily-publicized withdrawal of several batches of cheese contaminated with Listeria monocytogenes from the market in December 2015. As a consequence, the stakeholders in the sector are actively researching solutions that would address the risks of Listeria better. Some companies that specialize in the production and marketing of microorganisms have proposed microbial biopreservative cultures with anti-Listeria properties. As an example, it is known that certain strains of Lactobacillus plantarum have an anti-Listeria activity. One strain of Lactobacillus plantarum is already being marketed under the trade name HOLDBAC® in the capacity of a biopreservation fermenting agent.

However, despite the use of these biopreservative cultures, Listeria monocytogenes is still present, very probably because the biopreservative cultures that have been proposed are conventional lactic bacteria which cannot provide optimal and long-lasting protection throughout the lifetime of the food product. Furthermore, not all industrialists use methods of this type, because the solutions proposed are not compatible with every production method and every type of cheese.

The anti-Listeria activity of strains of lactic bacteria of the species Carnobacterium maltaromaticum has already been described in the prior art. As an example, Afzal et al. (Food Microbiology 36(2013) 223-230) describe a strain of Carnobacterium maltaromaticum LMA28 that has an anti-Listeria monocytogenes activity.

However, until now, the biopreservative bacteria that exist on the market as well as the strains of Carnobacterium maltaromaticum described in the literature have all been identified and selected using a selection criterion based solely on the inhibiting capacity of Carnobacterium maltaromaticum in relation to a single strain of L. monocytogenes.

The strains selected using that criterion cannot meet the requirements and demands of the food industry, in particular in the production of cheese.

For the first time, the Inventors of the present invention have indicated the importance of robustness in anti-bacterial efficacy for a strain of Carnobacterium maltaromaticum and have developed three criteria that can be used to isolate and select strains which have high robustness.

The term “robustness” is defined here as a bacterium for which the antibacterial activity is maintained (1) even if it is inoculated at low concentrations, (2) even it if is inoculated belatedly compared with Listeria monocytogenes, and (3) if it inhibits a wide variety of strains of Listeria monocytogenes.

Until now, none of the strains of Carnobacterium maltaromaticum described in the prior art has satisfied all of these criteria. As a consequence, it is necessary to select novel strains of Carnobacterium maltaromaticum for use thereof for the purposes of biopreservation of a food product.

The objective of the present invention is to propose novel strains of Carnobacterium maltaromaticum.

For a strain of Carnobacterium maltaromaticum to be retained and considered to have satisfactory robustness, three selection criteria have been set. Three criteria are applied simultaneously in order to screen strains:
an anti-Listeria efficacy with a low inoculation rate, namely less than 10² CFU·mL⁻¹ of initial Carnobacterium maltaromaticum
an anti-Listeria efficacy even when inoculation of Carnobacterium maltaromaticum is belated compared with that of Listeria, even at a low inoculation rate,
a broad spectrum of activity in relation to a variety of strains of Listeria monocytogenes and Listeria.

Due to the fact that collections of Carnobacterium maltaromaticum are selected on the basis of the activity of a strain of Carnobacterium maltaromaticum against a specific strain of Listeria monocytogenes, it is impossible to predict whether a strain could exist which will meet all of the three criteria. Against all the odds, two strains of Carnobacterium maltaromaticum were isolated at the end of several campaigns for screening Carnobacterium maltaromaticum by simultaneously applying the above three criteria.

The invention also concerns a strain CNCM I-5242 deposited on 26 Sep. 2017 and a strain CNCM I-5243 deposited on 26 Sep. 2017 at the Collection Nationale de Cultures de Microorganismes [National Microorganism Culture Collection].

These two strains have an anti-Listeria monocytogenes activity that is greater than that of the strain of Lactobacillus plantarum marketed under the trade name HOLDBAC® (product number 1209325) as a biopreservative strain.

Even at an inoculation rate of less than 100 CFU·mL⁻¹, these two isolated strains exhibit an effective anti-Listeria activity.

Furthermore, these two strains have a significant anti-Listeria activity in relation to the majority of the strains of Listeria monocytogenes tested during the screening experiments.

Even under co-culture, i.e. during simultaneous inoculation with Listeria monocytogenes, a condition which corresponds to that which could genuinely be encountered in cheese production, these two strains could still significantly inhibit the growth of Listeria monocytogenes in the co-culture.

In addition, cheese production tests demonstrate that these strains do not inhibit the growth of lactic fermenting agents and ripening fermenting agents. Under certain conditions and types of production, the use of these strains could even provide the cheeses with a particular and agreeable flavor.

In another aspect, the present invention concerns a bacterial preparation comprising the strain CNCM I-5242 or the strain CNCM I-5243.

The term “bacterial preparation” means a packaged composition for the growth of Carnobacterium maltaromaticum, said composition comprising a viable strain of Carnobacterium maltaromaticum and components that are essential for the growth of the bacteria, such as a source of carbon and of energy, a source of potassium and of phosphorus, a source of calcium, a source of magnesium, lipids, proteins or amino acids.

Said bacterial preparation may comprise a culture medium or a culture medium extract. Said medium may be TSB-YE medium (Tryptic Soy Broth-Yeast Extract).

Said culture medium may be a synthetic medium. The term “synthetic medium” as used in the present invention means a medium into which individual components have been introduced which have been characterized and have undergone rigorous quantitative and qualitative checks.

Said culture medium may also be a natural medium. The term “natural medium” as used in the present invention means a medium that contains one or more natural compounds in its composition. Said natural medium may be milk, in particular pasteurized whole milk.

Said bacterial preparation may be in the liquid or solid form.

In one embodiment of the invention, the concentration of Carnobacterium maltaromaticum in said bacterial preparation of the invention may be from 10² CFU·mL⁻¹ to 10¹¹ CFU·mL⁻¹, in particular from 10⁵ CFU·mL⁻¹ to 10⁷ CFU·mL⁻¹, more particularly at least 10⁵ CFU·mL⁻¹, or at least 10⁶ CFU·mL⁻¹, or at least 10⁷ CFU·mL⁻¹, with respect to the total volume of the bacterial preparation.

In another embodiment of the invention, said bacterial preparation contains a quantity of Carnobacterium maltaromaticum of 10² UFC·g⁻¹ to 10¹¹ UFC·g⁻¹, in particular of 10⁵ UFC·g⁻¹ to 10⁷ UFC·g⁻¹, at least 10⁵ UFC·g⁻¹, or at least 10⁶ UFC·g⁻¹, or at least 10⁷ UFC·g⁻¹, with respect to the total weight of the bacterial preparation.

In a particular embodiment, said bacterial preparation is a liquid concentrate, a frozen concentrate, or a lyophilizate.

The term “concentrate” means a liquid containing a quantity of bacteria of more than 10⁵ CFU·mL⁻¹, in particular more than 10¹⁰ CFU·mL⁻¹, in particular more than 10¹¹ CFU·mL⁻¹, with respect to the total volume of concentrate.

The concentrate may be obtained by any conventional method, for example by centrifuging or by microfiltration of a culture of Carnobacterium maltaromaticum after suitable incubation.

The term “lyophilizate” means the product obtained after lyophilization of a culture of Carnobacterium maltaromaticum after suitable incubation.

The term “suitable incubation” means incubation in accordance with a conventional method in order to obtain an initial population of Carnobacterium maltaromaticum of close to 10⁷ CFU·mL⁻¹, with respect to the total incubation volume.

By way of example, the incubation of Carnobacterium maltaromaticum may be carried out at 30° C. for 48 hours in pasteurized whole milk.

In a particular embodiment, said bacterial preparation is a concentrate, optionally frozen, comprising pasteurized whole milk and at least 10⁷ CFU·mL⁻¹ of Carnobacterium maltaromaticum CNCM I-5242, with respect to the total volume of the bacterial preparation.

In another particular embodiment, said bacterial preparation is a concentrate, optionally frozen, comprising pasteurized whole milk and at least 10⁷ CFU·mL⁻¹ of Carnobacterium maltaromaticum CNCM I-5243, with respect to the total volume of the bacterial preparation.

In one embodiment of the invention, said bacterial preparation may further comprise at least one lactic fermenting agent and/or at least one ripening fermenting agent.

In accordance with one embodiment of the invention, said bacterial preparation is a protective culture of Carnobacterium maltaromaticum.

The term “protective culture of Carnobacterium maltaromaticum” means a composition containing live Carnobacterium maltaromaticum which may be added to food products in order to inhibit the growth of microorganisms which are pathogenic or toxic to humans or animals and are present in said food products, in order to reduce the risk of contamination of the food product by these pathogenic or toxic microorganisms.

In the context of the present invention, said pathogenic or toxic microorganism is a bacterium from the genus Listeria, in particular Listeria monocytogenes, Listeria innocua, Listeria ivanovii.

Said protective culture may be in the form of a liquid concentrate or in the form of a lyophilizate.

The present invention also concerns the use of Carnobacterium maltaromaticum, of the strain CNCM I-5242 or of the strain CNCM I-5243, or a bacterial preparation as described above, or a protective culture as described above, in the preparation of a food product.

More particularly, the strains of Carnobacterium maltaromaticum, the bacterial preparation or the protective culture described above are used in order to inhibit the growth of bacteria of the genus Listeria in a food product.

Said protective culture of the invention may be used in the production of fermented or non-fermented milk products, in particular in the production of cheeses or fermented milk.

In a particular embodiment, said food product is a fermented milk product, in particular cheeses, such as soft cheeses, hard cheeses or semi-soft cheeses, cottage cheese, goat's cheese, sheep's cheese or fermented milk.

Said protective culture of the invention may also be used in the production of other dairy products such as cream, ice-cream, butter or by-products derived from milk, such as lactoserum, casein or other prepared food products containing milk or milk constituents as an ingredient.

Said protective culture of the invention may also be used in the production of beverages, for example fruit juices, beverages obtained with mixtures of milk and fruit juice, soy milk, oat milk, rice milk or fermented plant products.

Said protective culture of the invention may also be used in the industrial production of cured products and delicatessen products.

The use of the protective culture of the invention during the production of the food products means that the growth of bacteria of the genus Listeria, which remain in a food product in small quantities, can be inhibited, therefore extending the shelf life of said food product.

The protective culture of Carnobacterium maltaromaticum in accordance with the present invention is added to the food products as a function of the product type.

For fermented milk products such as cheeses or fermented milk, the protective culture is added before or at the same time as the lactic fermenting agents, or after adding them.

For the other food products, the protective culture may be added at the end of the production line and in particular before packaging the food product.

By way of example, the protective culture of Carnobacterium maltaromaticum in accordance with the present invention is added to a liquid food product in order to obtain a level of 10² CFU·mL⁻¹ to 10¹¹ CFU·mL⁻¹, in particular of 10⁵ CFU·mL⁻¹ to 10⁷ CFU·mL⁻¹, more particularly at least 10⁶ CFU·mL⁻¹, in particular between 10⁶ CFU·mL⁻¹ and 10⁷ CFU·mL⁻¹, with respect to the total volume of said liquid food product; or added to a solid food product in order to obtain a level of 10² UFC·g⁻¹ to 10¹¹ UFC·g⁻¹, in particular of 10⁵ UFC·g⁻¹ to 10⁷ UFC·g⁻¹, at least 10⁵ UFC·g⁻¹, or at least 10⁶ UFC·g⁻¹, or at least 10⁷ UFC·g⁻¹, with respect to the total weight of said solid food product, during the shelf life of said food product.

In another aspect, the invention concerns a method for the preparation of a cheese, in particular a soft cheese or a semi-soft cheese, using the CNCM I-5242 strain or the CNCM I-5243 strain or a bacterial preparation containing one of the two strains.

Said method comprises:
inoculating with the Carnobacterium maltaromaticum bacterial strain CNCM I-5242 or CNCM I-5243 or a bacterial preparation containing the CNCM I-5242 or CNCM I-5243 strain into the milk or during transformation of the milk.

In accordance with the method of the invention, the Carnobacterium maltaromaticum bacteria may be inoculated into the milk, into the lactoserum, into the brine, or directly into the curd.

In accordance with the method of the invention, when inoculation is carried out onto the curd, it may be carried out by sprinkling or spraying a liquid preparation of Carnobacterium maltaromaticum onto the curd.

The term “inoculation during transformation of the milk” in the production of cheese means inoculated during storage, coagulation, draining, milling, pressing, salting, ripening or washing.

In accordance with the method of the invention, when the inoculation is carried out into the milk, the inoculation may be carried out with or without cold maturation of the milk.

Furthermore, inoculation of the Carnobacterium maltaromaticum bacteria of the invention into the milk may be carried out before or simultaneously with inoculation of the lactic fermenting agents.

In another embodiment, the inoculation of Carnobacterium maltaromaticum is carried out 0 to 8 hours before that of the lactic fermenting agents.

The milk used in said method may be milk, whole or otherwise, raw or pasteurized, or milk which has undergone a heat treatment.

The term “pasteurized whole milk” means whole milk which has undergone a heat treatment at 71.5° C. for 15 seconds, then been cooled rapidly to 4° C.

In one embodiment of the method, the milk is inoculated with Carnobacterium maltaromaticum in an amount of 10² CFU·mL⁻¹ to 10¹¹ CFU·mL⁻¹, in particular from 10⁶ CFU·mL⁻¹ and 10⁷ CFU·mL⁻¹, with respect to the volume of the milk.

The present invention also concerns a method for reducing the microbial contamination during the process for the production of a fermented milk product, comprising the addition of an inhibiting quantity of Carnobacterium maltaromaticum strain CNCM I-5242 or CNCM I-5243 or a bacterial preparation containing the CNCM I-5242 or CNCM I-5243 strain.

The present invention also proposes a method for reducing contamination by bacteria of the genus Listeria during a process for the production of a fermented milk product, characterized in that it comprises the addition of a quantity of 10² CFU·mL⁻¹ to 10¹¹ CFU·mL⁻¹, in particular of 10⁶ to 10⁷ UFC·mL⁻¹, with respect to the volume of the milk product, of Carnobacterium maltaromaticum strain CNCM I-5242 or CNCM I-5243, or a bacterial preparation comprising these strains.

The various aspects of the invention are presented in more detail below in the figures and the examples.

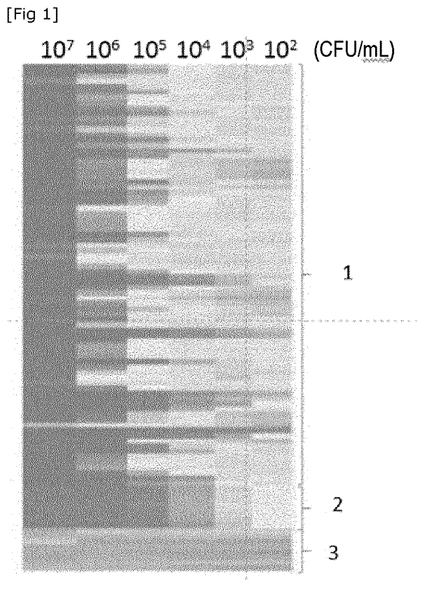

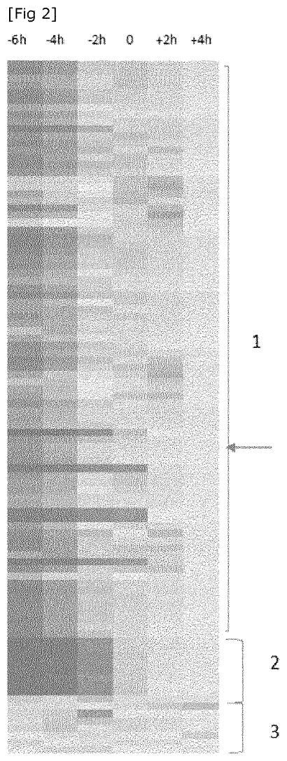

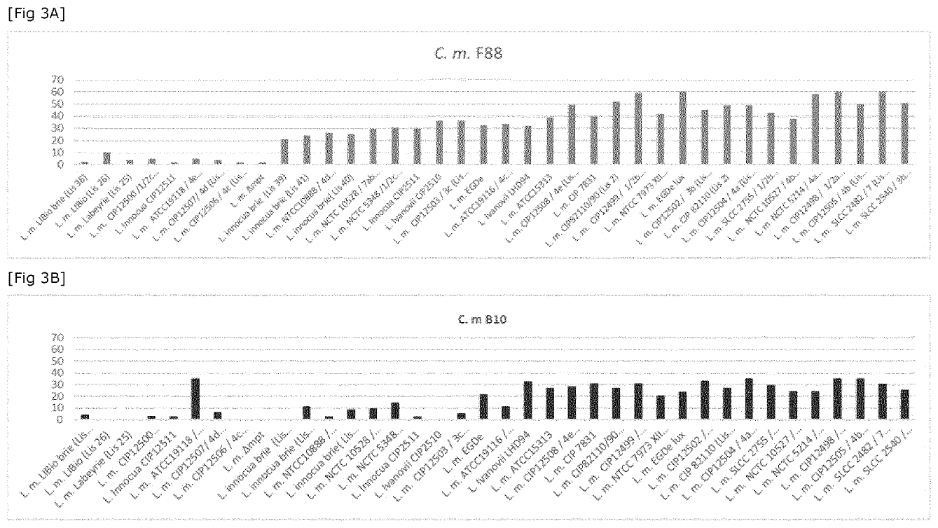

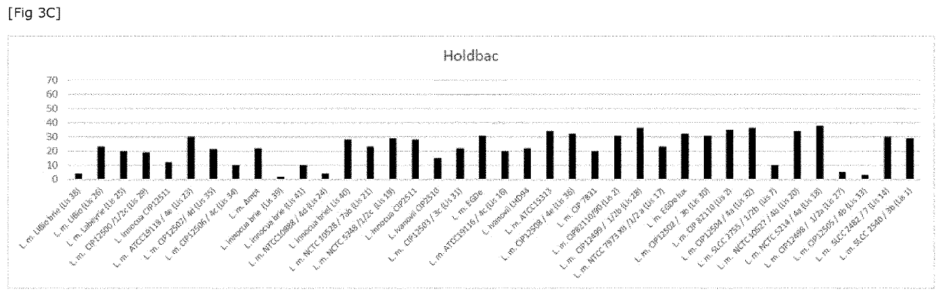

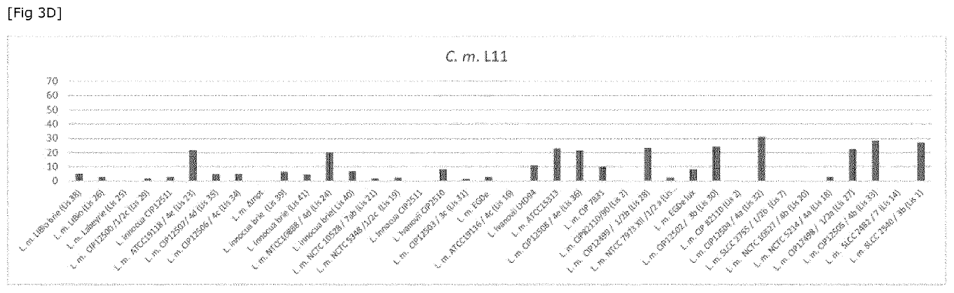

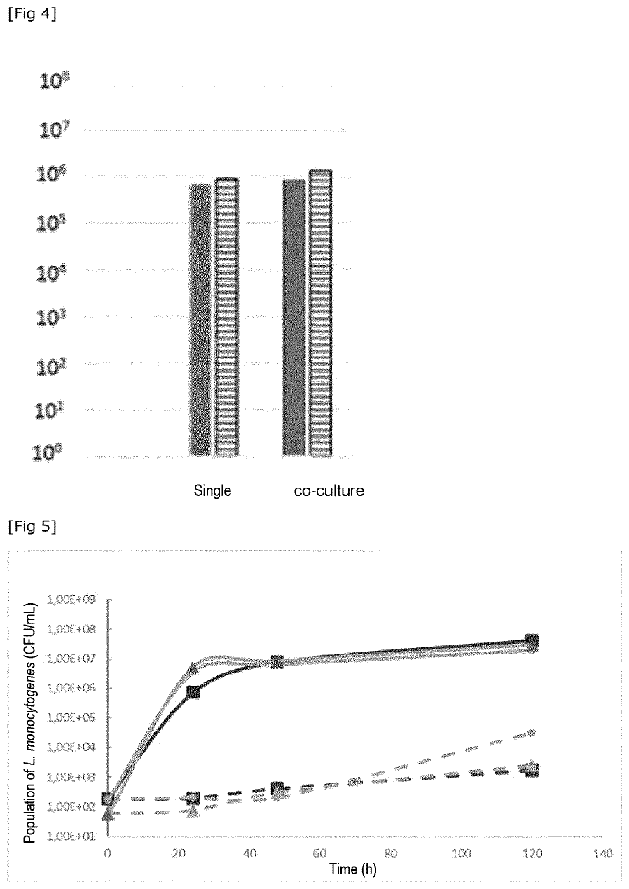

### FIGURES

[FIG. 1] Influence of inoculation rate on the anti-Listeria monocytogenes properties of a collection of 74 strains of Carnobacterium maltaromaticum into UHT whole milk (ultra high temperature). Each strain of Carnobacterium maltaromaticum was co-cultivated with Listeria monocytogenes EGDelux in different inoculation rates. The growth of Listeria monocytogenes EGDe Lux was specifically monitored by bioluminescence. An optimal growth of Listeria monocytogenes EGDe Lux is shown in pale gray, an absence of the detection of growth of Listeria monocytogenes EGDe Lux is shown in dark gray, an intermediate growth is shown in the form of a color gradient ranging from dark gray to pale gray. The lines in section “1” correspond to the results obtained for the Carnobacterium maltaromaticum/Listeria monocytogenes EGDe Lux strain pair in different inoculation rates. The lines in section “2” are the results obtained for the positive controls, corresponding to the co-culture of Listeria monocytogenes EGDe in the presence of Listeria monocytogenes EGDe Lux. The lines in section “3” are the results for the negative controls, obtained in the presence of Listeria monocytogenes EGDe Lux.

[FIG. 2] Influence of time of inoculation of Carnobacterium maltaromaticum on the anti-Listeria monocytogenes properties of a collection of 73 strains of Carnobacterium maltaromaticum in UHT whole milk. Each strain of Carnobacterium maltaromaticum was inoculated at different times compared with Listeria monocytogenes. “−6 h”, “−4 h”, “−2 h” respectively signify the inoculation of Carnobacterium maltaromaticum 6 h, 4 h, 2 h before inoculating with Listeria monocytogenes. “0” signifies the inoculation of Carnobacterium maltaromaticum at the same time as Listeria monocytogenes. “+2 h”, “+4 h” respectively signify the inoculation of Carnobacterium maltaromaticum 2 h, 4 h after the inoculation of Listeria monocytogenes. An optimal growth of Listeria monocytogenes is shown in pale gray, an absence of the detection of growth of Listeria monocytogenes is shown in dark gray, an intermediate growth is shown in the form of a color gradient ranging from dark gray to pale gray. One line represents the results obtained for the Carnobacterium maltaromaticum/Listeria monocytogenes strain pair inoculated at different time delays. The arrow indicates the line corresponding to the strain LMA28 which has been described in the prior art. The lines in section “1” correspond to the obtained for results the Carnobacterium maltaromaticum/Listeria monocytogenes strain pair at different inoculation rates. The lines in section “2” are the results obtained for the positive controls. The lines in section “3” are the results for the negative controls.

[FIG. 3A] Inhibiting activity of Carnobacterium maltaromaticum CNCM I-5242 (in the experiments below, represented by F88) in relation to 38 strains of Listeria by measurement of inhibition zones (mm). The ordinate represents the diameters of the inhibition zones measured by the double layer Petri dish method.

[FIG. 3B] Inhibiting activity of Carnobacterium maltaromaticum CNCM I-5243 (in the experiments below, represented by B10) in relation to 38 strains of Listeria by measurement of inhibition zones (mm). The ordinate represents the diameters of the inhibition zones measured by the double layer Petri dish method.

[FIG. 3C] Inhibiting activity of the bioprotective strain Lactobacillus plantarum Holdbac in relation to 38 strains of Listeria by measurement of inhibition zones (mm). The ordinate represents the diameters of the inhibition zones measured by the double layer Petri dish method.

[FIG. 3D] Inhibiting activity of Carnobacterium maltaromaticum L11 in relation to 38 strains of Listeria by measurement of inhibition zones (mm). The ordinate represents the diameters of the inhibition zones measured by the double layer Petri dish method.

[FIG. 4] Population levels for the CNCM I-5242 strain (in the experiments below, represented by F88) of Carnobacterium maltaromaticum in single culture or in co-culture with Listeria monocytogenes at the start time (solid bar) and after 48 h of growth (hatched bar) at 14° C. The ordinate represents the population of Carnobacterium maltaromaticum (CFU·mL⁻¹).

[FIG. 5] Change in populations over time, after growth at 14° C., of Listeria monocytogenes EGDe (triangle), Listeria monocytogenes Lis 1 (square) and Listeria monocytogenes Lis 34 (circle) in single culture (solid line) or in the presence of strains of Carnobacterium maltaromaticum, Carnobacterium maltaromaticum C. m. CNCM I-5242 (broken line). The ordinate represents the population of Carnobacterium maltaromaticum (CFU·mL⁻¹). The abscissa represents the incubation time (hours).

[FIG. 6] Growth of Carnobacterium maltaromaticum C. m. CNCM I-5242 in the absence (1) and in the presence of 8 commercial fermenting agents at the start time (pale gray) and after 24 h of co-culture (dark gray), 1: Control, 2: Fermenting agent Lc2a, 3: Fermenting agent Lc2b, 4: Fermenting agent lc3, 5:

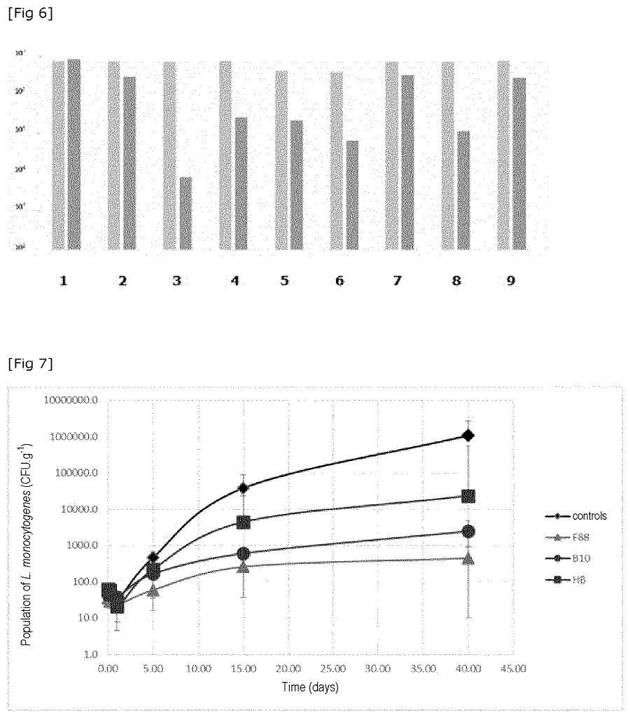

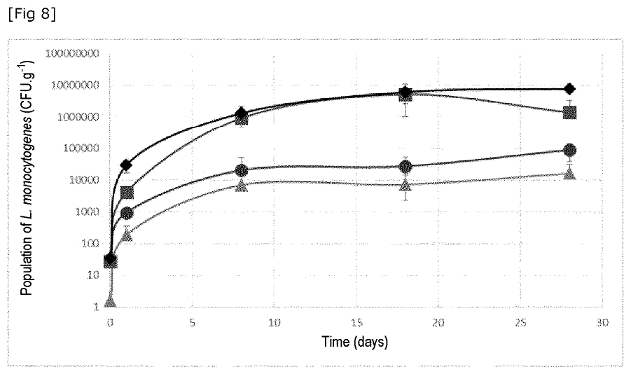

Fermenting agent 5t, 6: Fermenting agent Lc-St, 7: Fermenting agent Lc-St-Lb, 8: Fermenting agent M.A-Lb, 9: Fermenting agent F.D.

[FIG. 7] Comparison of the change in the mean values for the populations of Listeria monocytogenes over time for the 3 soft cheese production series in the “controls” tests with Listeria monocytogenes alone with no protective culture (represented by a diamond) or in the presence of the various protective cultures Carnobacterium maltaromaticum CNCM I-5242 (in the experiments below, represented by “F88”, represented by a triangle), Carnobacterium maltaromaticum CNCM I-5243 (in the experiments below, represented by “B10”, represented by a spot), and for the commercial culture Holdbac® (in the experiments below, represented by “HB”, represented by a square). The ordinate represents the population of Listeria monocytogenes (UFC·g⁻¹). The abscissa represents the time after inoculation (days).

[FIG. 8] Comparison of the change in the mean values for the populations of Listeria monocytogenes during Saint Nectaire type production in the “controls” test (represented by a diamond) or in the presence of the protective cultures Carnobacterium maltaromaticum CNCM I-5242 (in the experiments below, represented by “F88”, represented by a triangle) and Carnobacterium maltaromaticum CNCM I-5243 (in the experiments below, represented by “B10” represented by a spot) and for the commercial culture Holdbac® (in the experiments below, represented by “HB”, represented by a square). The ordinate represents the population of Listeria monocytogenes (UFC·g⁻¹). The abscissa represents the time after inoculation (days).

### EXAMPLES

### Example 1: Selection of Strains

Strains of Carnobacterium maltaromaticum with an anti-Listeria monocytogenes activity were initially identified using a single strain of Listeria monocytogenes.

Once identified, these strains constituted a collection for the subsequent screening.

Said screening was carried out by simultaneously applying the 3 criteria explained above:
a low inoculation rate, namely less than 10² UFC/mL of initial Carnobacterium maltaromaticum,
an anti-Listeria efficacy, even with belated inoculation of Carnobacterium maltaromaticum compared with that of Listeria, even at a low inoculation rate, even at a low inoculation rate
a broad spectrum of activity as regards a variety of strains of Listeria monocytogenes and of Listeria.

### Method and Apparatus

1. Preparation of Optimized Pre-Culture of Strains of Carnobacterium maltaromaticum

A collection of Carnobacterium maltaromaticum strains was constituted in the Inventors' laboratories by several campaigns of isolation from cheeses, human blood and raw milk. Screening of this collection enabled strains with anti-Listeria monocytogenes properties to be identified.

A fraction from a cryotube stored at −80° C. containing a strain of Carnobacterium maltaromaticum to be tested was removed in a sterile manner with the aid of a sterile loop and stabbed into 10 mL of TSB-YE broth, incubated at 30° C. for 24 h. The bacterial culture was centrifuged (3000 rpm, 10 min, 4° C.) then washed twice with sterile physiological water. The bacterial pellet was taken up in 10 mL of pasteurized whole milk, then was introduced into 100 ml of pasteurized whole milk so as to obtain an initial population of close to 10⁷ cells·mL⁻¹. The inoculated milk was incubated at 30° C. in a water bath, with stirring. The growth of the strains of Carnobacterium maltaromaticum was monitored by counting on TSA-YE medium for 48 h. Pre-culturing the strains enabled large quantities of viable cells to be obtained which were metabolically active without a latency phase.

2. Listeria monocytogenes Strains

The strains of Listeria monocytogenes used in the experiments originated from public collections (American Type Culture Collection—USA (ATCC); Collection de l'Institut Pasteur—France (CIP); National Collection of Type Culture—Great Britain (NCTC); Seeliger Listeria Culture Collection—Germany), from research laboratories (EGDe, EGDe Lux, Δmpt) and from the Inventors' laboratories. The test strains included 31 strains of Listeria monocytogenes, 2 strains of Listeria ivanovii and 5 strains of Listeria innocua (Table 1 below). The strains were revived from cultures frozen at −80° C. (TSB-YE culture cryotubes+15% glycerol) in a TSB-YE culture broth and incubated at their optimal growth temperature, i.e. 30° C., for 24 h.

The Listeria monocytogenes EGDe Lux strain was used for the bioluminescence experiment.

TABLE 1
List of strains of Listeria selected for the study of
the anti-Listeria activity of strains of Carnobacterium maltaromaticum
(American Type Culture Collection - USA (ATCC); Collection
de l'Institut Pasteur - France (CIP); National Collection
of Type Culture - Great Britain (NCTC); Seeliger Listeria
Culture Collection - Germany)

| | | | Year of |
|---|---|---|---|
| Names | Origin/serotype | Code | isolation |
| Listeria | ATCC 19118/4e | Lis 23 | 1995 |
| monocytogenes | ATCC 19116/4c | Lis 16 | 1995 |
| | ATCC 15313 | | — |
| | CIP 82110/90 | Lis 2 | 1994 |
| | CIP 12498/½a | Lis 27 | 1994 |
| | CIP12499/½b | Lis 28 | 1994 |
| | CIP 12500/½c | Lis 29 | 1994 |
| | CIP 12502/3b | Lis 30 | 1994 |
| | CIP 12503/3c | Lis 31 | 1994 |
| | CIP 12504/4a | Lis 32 | 1994 |
| | CIP 12505/4b | Lis 33 | 1994 |
| | CIP 12506/4c | Lis 34 | 1994 |
| | CIP 12507/4d | Lis 35 | 1994 |
| | CIP 12508/4e | Lis 36 | 1994 |
| | CIP 7831 | | — |
| | CIP 82110 | | — |
| | NTCC 7973 XII | Lis 17 | 1995 |
| | NCTC 5214/4a | Lis 18 | 1995 |
| | NCTC 5348/½c | Lis 19 | 1995 |
| | NITC 10527/4b | Lis 20 | 1995 |
| | NCTC 10528/7ab | Lis 21 | 1995 |
| | NTCC10888/4d | Lis 24 | 1995 |
| | SLCC 2755/½b | Lis 7 | 1995 |
| | SLCC 2540/3b | Lis1 | 1995 |
| | SLCC 2482/7 | Lis 14 | 1995 |
| | LIBio brie | Lis 38 | 1989 |
| | LIBio Labeyrie | Lis 25 | 1994 |
| | Lis LIBio | Lis 26 | |
| | Δ mpt* | | — |
| | EGDe | | — |
| | EGDe Lux | | — |
| Listeria innocua | CIP 12511 | | — |
| | CIP 2511 | | — |
| | LIBio brie | Lis 41 | 1989 |
| | LIBio brie | Lis 40 | 1989 |
| | LIBio brie | Lis 39 | 1989 |
| Listeria ivanovii | CIP 2510 | | — |
| | LHD94 | | — |

*strain modified so as to be resistant to class IIa bacteriocins, anti-Listeria monocytogenes

### 3. Control Strains

A HOLDBAC® Lactobacillus plantarum strain known for its anti-Listeria activity and already marketed as a biopreservation fermenting agent was used as a control in order to compare the anti-Listeria activity of strains of Carnobacterium maltaromaticum with those of a commercial bioprotection fermenting agent.

The LMA28 Carnobacterium maltaromaticum strain described by Afzal et al. (Food Microbiology 36(2013) 223-230) was also used in some experiments as a control.

4.1 Anti-Listeria Activity of Strains of Carnobacterium maltaromaticum

The anti-Listeria activity of strains of Carnobacterium maltaromaticum was determined using the double layer method in a Petri dish containing the gel medium TSA-YE (Biomerieux, Marcy-l'Etoile, France). A colony of Carnobacterium maltaromaticum was inoculated by stabbing the surface of a TSA-YE gel medium (Biomerieux, Marcy-l'Etoile, France) poured into a Petri dish, then incubated at 30° C. until a colony appeared (24 h). The strains of Listeria were stabbed into TSB-YE broth and incubated at 30° C. for 24 h. The strains of Listeria monocytogenes were diluted to one hundredth in supercooled TSA-YE gel medium at 50° C., in a manner such as to obtain a concentration of approximately 10⁶ cells/mL. A volume of 15 mL of gel medium was poured onto the surface of TSA-YE gels inoculated with the strains of Carnobacterium maltaromaticum (or Holdbac Listeria plantarum). The Petri dishes were then incubated at 30° C. for 24 h. The inhibiting activity was evaluated by measuring the diameters of the inhibition zones surrounding each productive zone (in millimeters).

4.2 Anti-Listeria Activity of Strains of Carnobacterium maltaromaticum Using a Bioluminescence Test

The initial pre-cultures of the different strains of Carnobacterium maltaromaticum were prepared as indicated above in pasteurized whole milk. The Listeria monocytogenes EGDe Lux strain was revived from a culture frozen at −80° C. (TSB-YE culture cryotubes+15% glycerol) in a TSB-YE culture broth and incubated at 30° C. for 24 h. Using a robot (TECAN), 96-well plates were filled with 160 μL of whole milk or semi-skimmed milk and inoculated with 20 μL of the pre-culture of Listeria monocytogenes EGDe lux diluted to 1/1000000. Serial tenfold dilutions of the cultures of Carnobacterium maltaromaticum were carried out in whole milk or semi-skimmed milk. A volume of 20 μL of the first four dilutions was deposited in wells, following a predefined plating plan. Tests with 24 h pre-cultures of Carnobacterium maltaromaticum in TSB-YE medium as well as cultures of Listeria monocytogenes EGDe Lux cultured in the absence of strains of Carnobacterium maltaromaticum served as controls. They were carried out in triplicate. The bioluminescence was measured using an Infinite 200 Pro microplate reader equipped with a luminescence module (TECAN group Ltd. Mânnedorf, Switzerland). The measurements were carried out on opaque white microplates.

### Results

Influence of Inoculation Rate of Carnobacterium maltaromaticum

The influence of inoculation rate on the anti-Listeria monocytogenes properties of Carnobacterium maltaromaticum was evaluated by luminescence. Each strain of Carnobacterium maltaromaticum was co-cultivated with Listeria monocytogenes at different inoculation rates. The growth of Listeria monocytogenes was specifically monitored by luminescence.

A collection of strains of Carnobacterium maltaromaticum to be screened was analyzed using this method in order to select strains that had an anti-Listeria monocytogenes activity even at a low inoculation rate.

This experiment shows that at a high concentration, all of the strains of Carnobacterium maltaromaticum are capable of inhibiting Listeria monocytogenes. In contrast, at low concentrations of Carnobacterium maltaromaticum and Listeria. monocytogenes, conditions which are more representative of the levels of contamination with Listeria monocytogenes in foods, only a few strains of Carnobacterium maltaromaticum, including the strain CNCM I-5242 (in the experiments below, represented by F88) and the strain CNCM I-5243 (in the experiments below, represented by B10), still inhibit Listeria monocytogenes. The result is shown in FIG. 1.

Reduced Time Delay for the Inoculation of Carnobacterium maltaromaticum

The influence of delaying the inoculation of Carnobacterium maltaromaticum on the anti-Listeria monocytogenes properties was evaluated by luminescence.

The carnobacteria were inoculated either 6 h, 4 h, 2 h before the inoculation of Listeria monocytogenes, or at the same time as the inoculation of Listeria monocytogenes, or 2 h or 4 h after the inoculation of Listeria monocytogenes.

The result shows that when the strains of Carnobacterium maltaromaticum were inoculated 6 h before Listeria monocytogenes, many strains could inhibit Listeria monocytogenes.

In contrast, the shorter the time delay, the greater the reduction in the number of strains that are capable of inhibiting Listeria. Thus, when the strains were inoculated at the same time (condition “0”) only 4 strains, including the strain CNCM I-5242 (in the experiments below, represented by F88) and the strain CNCM I-5243 (in the experiments below, represented by B10), still inhibited Listeria monocytogenes effectively (FIG. 2).

FIG. 2 shows that the strain LMA28 mentioned in the prior art has a fairly low inhibiting activity under the test conditions.

Anti-Listeria Activity of Carnobacterium maltaromaticum in Relation to Different Strains of Listeria

In order to determine whether a strain of Carnobacterium maltaromaticum has a broad anti-Listeria activity spectrum, the anti-Listeria activity of said strain was evaluated in relation to a collection of 31 strains of Listeria monocytogenes, 2 strains of Listeria ivanovii and 5 strains of Listeria innocua (Table 1 above). A Lactobacillus plantarum HOLDBAC® strain which is known for its anti-Listeria activity and already marketed as a biopreservation fermenting agent was used as the control in order to compare the anti-Listeria activity of the strains of Carnobacterium maltaromaticum with those of a commercial bioprotection fermenting agent.

The two strains of the present invention, CNCM I-5242 and CNCM I-5243, exhibited a strong anti-Listeria activity in relation to the majority of the strains of Listeria tested in this experiment. For some strains of Listeria, the CNCM I-5242 strain (in the experiments below, represented by F88) of the present invention even exhibits a better inhibiting activity compared with the Lactobacillus plantarum HOLDBAC® strain (FIGS. 3A, 3B, 3C).

By way of a contrasting example, FIG. 3D shows the result obtained for a strain of Carnobacterium maltaromaticum represented by L11 which was tested under the same experimental conditions. Although this strain was initially retained in the test by bioluminescence because of its inhibiting activity for Listeria monocytogenes EGDe Lux, this strain did not exhibit a sufficient anti-Listeria activity in relation to the majority of the Listeria strains tested in this experiment.

### Example 2: Validation of the Anti-Listeria monocytogenes Activity of Strains of Carnobacterium maltaromaticum by Co-Culture in a Milk Medium

It was shown that the strains of Listeria monocytogenes have different levels of sensitivity, as shown by inhibition diameters that varied from 1 to 64 mm. In the cheesemaking industry, the growth of all strains of Listeria monocytogenes, irrespective of their origin, has to be inhibited. During the earlier studies, the concentration of Listeria used in order to determine the activity spectrum was high, close to 10⁶ cells·mL⁻¹. In cheese production, levels of contamination with Listeria are low, below 100 cells·mL⁻¹. The earlier tests were carried out in a laboratory environment, while the inhibiting strains have to be effective in a milk medium.

In order to take all of these factors into account, it is necessary to:
analyze the inhibiting efficacy of the selected strains of Carnobacterium maltaromaticum in relation to the growth of Listeria monocytogenes in milk, in the presence of levels of contamination close to those encountered in cheeses;
determine whether the least sensitive strains of Listeria monocytogenes are inhibited when low inoculation rates are used for Listeria.

The aim of these studies was to analyze the inhibiting efficacy of selected strains of Carnobacterium maltaromaticum in relation to the growth of Listeria monocytogenes in milk, in the presence of levels of contamination close to those encountered in cheeses and to determine whether the least sensitive strains of Listeria monocytogenes are inhibited when low inoculation rates are employed for Listeria. The three strains of Listeria monocytogenes, Listeria monocytogenes CIP 12506 (Lis 1), Listeria monocytogenes EGDe and Listeria monocytogenes SLCC 2540 (Lis 34), representative of 3 levels of sensitivity-respectively high, medium and low-were used for these studies.

Method and apparatus: The CNCM I-5242 strain (in the experiments below, represented by F88) of Carnobacterium maltaromaticum was pre-cultured for 48 h in pasteurized whole milk (see section above). The three strains of Listeria monocytogenes, Listeria monocytogenes CIP 12506 (Lis 1), Listeria monocytogenes EGDe and Listeria monocytogenes SLCC 2540 (Lis 34) were revived from a culture that had been frozen at −80° C. (TSB-YE culture cryotubes+15% glycerol) in a TSB-YE culture broth and incubated at 30° C. for 24 h. The bacterial pellets were then taken up in 10 mL of pasteurized whole milk after washing twice in physiological water. A volume of 1 mL of Carnobacterium maltaromaticum cultures was used to inoculate 100 mL of pasteurized whole milk to which each of the strains of Listeria monocytogenes, diluted so as to obtain an initial population of close to 50 CFU·mL⁻¹, had been added. Culture controls were produced with an absence of each of the strains of Listeria monocytogenes and with an absence of the Carnobacterium maltaromaticum strain. The populations of Listeria monocytogenes were enumerated by counting the colonies obtained after suspension/dilution of the sample in tryptone salt and spreading onto PALCAM medium incubated for 24 h at 30° C. The populations of Carnobacterium maltaromaticum were counted after 48 h employing the same technique by using the selective medium MCM (Medium for Carnobacterium Maltaromaticum); incubated for 24 h at 25° C. (Edima et al., 2007 J. Food Microbiol., 67:516-521). The populations of Listeria monocytogenes were determined over 120 h after incubation at 14° C. (cold pre-maturation temperature).

Result

The presence of the different strains of Listeria monocytogenes did not modify the growth of Carnobacterium maltaromaticum (FIG. 4). The population of Carnobacterium maltaromaticum remained stable during cultures at 14° C. after 48 h. After 120 h of culture, the population of Carnobacterium maltaromaticum increased by approximately log 0.5 to 1 log during the incubations at 14° C.

At 14° C., the 3 strains of Listeria monocytogenes develop on milk, reaching populations comprised between 5×10⁵ and 5×10⁶ CFU·mL⁻¹ in 24 h. The population levels were of the order of 5×10⁷ CFU·mL⁻¹ after 120 h of growth. In co-culture with the Carnobacterium maltaromaticum C. m CNCM I-5242 (F88) strain, the growth of 3 strains of Listeria monocytogenes was inhibited for at least 48 h, staying at population levels identical to the initial levels. After 120 h, the population levels of Listeria monocytogenes were log 3 to 4 lower than those of the controls (FIG. 5).

### Example 3: Compatibility Study for Strains of Carnobacterium maltaromaticum with Lactic Bacteria and Acidification Fermenting Agents

The use of a processing aid must not have a major impact on the acidification phase which is implemented by the lactic bacteria which are present in the composition of the acidification fermenting agents. Likewise, the lactic bacteria must not inhibit the growth of Carnobacterium maltaromaticum cultures so that they can continue to develop during the cheese ripening phase.

Il is important to study the impact of lactic bacteria on the viability of strains of Carnobacterium maltaromaticum as well as the impact of these strains of Carnobacterium maltaromaticum on the lactic bacteria, and in particular on the acidification kinetics of the fermenting agents.

Impact of Fermenting Agents on the Growth of Strains of Carnobacterium maltaromaticum in Co-Culture

The impact of fermenting agents on the viability of the strains of Carnobacterium maltaromaticum was analyzed during co-culture.

The fermenting agents used are listed in Table 2 below.

TABLE 2
References, composition and commercial use of the different fermenting agents used

| | Strains of lactic bacteria | | | | | | | |
|---|---|---|---|---|---|---|---|---|
| | Lactococcus lactis subsp. | | | | | | | |
| | | | lactis biovar | Sreptococcus | Lactobacillus | | | |
| Fermenting agents | cremoris | lactis | diacetylactis | thermophillus | helveticus | lactis | Leuconostoc | Use |
| Fermenting agent Lc2a: Choozit ® | X | X | | | | | | soft cheese, hard |
| MA 16 lyophilized 25DCU | | | | | | | | cheese, pressed cheese, |
| (homofermentative mesophile - | | | | | | | | quark types, sour cream, |
| Danisco) | | | | | | | | cottage cheese |
| Fermenting agent Lc2b: Choozit ® | X | X | | | | | | soft cheese, hard |
| MA 14 lyophilized 50DCU | | | | | | | | cheese, pressed cheese, |
| (homofermentative mesophile: | | | | | | | | quark types, sour cream, |
| Danisco) | | | | | | | | cottage cheese |
| Fermenting agent Lc3: PAL ® 122 | X | X | X | | | | | soft cheese, semi-soft |
| D2U (heterofermentative mesophile - | | | | | | | | cheese, hard cheese, sour |
| Standa) | | | | | | | | cream, cottage cheese |
| Fermenting agent St: Choozit ® TA | | | | X | | | | hard cheese, pressed |
| 60 lyophilized 25DCU (thermophilic | | | | | | | | cheese, asiago, quark |
| fermenting agent - Danisco) | | | | | | | | types, sour cream |
| Fermenting agent Lc - St: | X | X | X | X | | | | soft cheese, semi-soft |
| Choozit ® MA 4001 lyophilized | | | | | | | | cheese, quark types, |
| 5DCU (homofermentative | | | | | | | | sour cream |
| mesophile - Danisco) | | | | | | | | |
| Fermenting agent Lc - St - Lb: | X | X | X | X | X | X | | hard cheese, |
| PAL ® cheese 114 D2U (mesophile | | | | | | | | pressed cheese |
| and thermophile - Standa) | | | | | | | | |
| Fermenting agent FD: MSP | X | X | X | | | | X | soft cheese, blue, |
| lyophilized 10U CHR Hansen | | | | | | | | pressed cheese |
| Fermenting agent M.A.: flavor | | | | | | | | |
| fermenting agent | | | | | | | | |

Method and apparatus: The Carnobacterium maltaromaticum CNCM I-5242 strain was pre-cultured for 48 h in pasteurized whole milk (see section above). A quantity of 0.05 to 0.08 g of lyophilized fermenting agent was cultured in 10 mL of pasteurized whole milk for 24 h at 30° C. After growth, serial tenfold dilutions, to a dilution of 10⁹, of this culture were carried out in pasteurized whole milk and incubated for 18 to 24 h at 30° C. The culture tube preceding the last tube which had not caused coagulation of the milk was retained in order to inoculate the co-culture. A volume of 40 mL of pasteurized whole milk was inoculated with a volume of 1 mL of Carnobacterium maltaromaticum pre-culture and 0.1 ml of fermenting agent culture. Milk inoculated with only the fermenting agent or the Carnobacterium maltaromaticum culture acted as the control. It was all incubated for 24 h at 30° C. The bacterial populations of Carnobacterium maltaromaticum were determined after spreading the inoculum onto MCM selective medium incubated for 48 h at 25° C. in accordance with the suspension/dilution technique.

### Results:

Although the final population of Carnobacterium maltaromaticum was affected by the presence of the fermenting agents (FIG. 6), for the whole set of the strains of fermenting agents tested, however, the cultures of Carnobacterium maltaromaticum remained viable after prolonged contact with the fermenting agents.

### Example 4: Impact of Protective Cultures on the Growth of Listeria monocytogenes During Cheese Production

### Preparation of Protective Cultures

For each bacterial strain, starting from a cryotube stored at −20° C., 5 tubes of 10 mL of TSBYE were prepared and noted with the number of the strain, from a to e. After centrifuging and washing the cells with physiological water, each of these tubes was used to inoculate a 100 mL flask of pasteurized whole milk denoted a to e. After growth, each flask was frozen in three Falcon tubes respectively containing 50 mL, 50 mL and 10 mL of bacterial culture. These tubes were stored at −20° C. The anti-bacterial activity of the different batches of protective cultures in relation to the mixture of strains of Listeria monocytogenes which were used during the cheese production tests was verified.

### Protocol for the Production of Soft Cheese

Three series of two productions of 20 soft cheeses were produced simultaneously. One production corresponded to 10 vats of 5 L of milk (2 cheeses of 250 g/vat), including 8 test vats with Listeria monocytogenes and the provided protective cultures (indicated dose) and 2 control vats with Listeria monocytogenesalone with no protective culture. The milk used was pasteurized and standardized milk (MF/PC=1, MF: milk fats, PC: protein content). The fermenting agents used for soft cheese techniques were Flora Danica (FD, Table 2) and the RNA fermenting agent.

The strains of Carnobacterium maltaromaticum (CNCM I-5242 (in the experiments below, represented by F88) and CNCM I-5243 (in the experiments below, represented by B10)) were stored in the ready-to use frozen form for each of the strains to be tested. The mixture of 5 strains of Listeria monocytogenes was constituted by the strains Scott A, CA2165 Fromage-CA Wisconsin E. Ryser, SN167 (strain isolated from St-Nectaire cheese), Lal65 (strain isolated from Laguiole cheese) and PL1547. The 5 strains (individually in BHI (brain heart infusion)) were pre-cultured. The mixture of the 5 strains was counted on Palcam medium at 37° C., and stored at −20° C. in 1.5 mL aliquots. A pre-test was validated in order to verify the level of growth of Listeria monocytogenes in accordance with this technique with two inoculation levels of 50 and 100 UFC·mL⁻¹.

The commercial protective culture Holdbac® (in the experiments below, represented by HB) was used in accordance with the manufacturer's recommendations in order to compare the results for the protective cultures of Carnobacterium maltaromaticum with those obtained with this commercial culture.

Inoculation of the mixture of Listeria monocytogenes at 50 UFC·mL⁻¹ and of Carnobacterium

Carnobacterium maltaromaticum (10⁶-10⁷ UFC·mL⁻¹) was added to milk on D-1 (start of cold maturation=T0). Inoculation of the Flora Danica and RNA fermenting agents was carried out at half cold maturation (at T8h D-1) in an amount of 1%.

Surface spraying of Geotricum and Pencillium candidum tool place after salting.

### Timing of Sampling:

T0 (D-1)=after inoculation of protective cultures and Listeria=30 ml of milk
T8 h (D-1)=30 mL milk
T20 h (D0)=end of cold maturation=30 mL milk
−T5 d=½ cheese: 25 g for counting of Listeria monocytogenes
−T15 d=½ cheese: 25 g for counting of Listeria monocytogenes
—T40 d=½ cheese: 25 g for counting of Listeria monocytogenes

The Listeria monocytogenes were counted on a mixture of curd and crust.

The pH of the samples that were taken was also measured.

### Results:

The appearance of all of the cheeses produced during the experiment was correct throughout production. All of the cheeses had the same appearance.

The pH of the cheeses produced and the dry extracts obtained were correct throughout production.

The population numbers for Listeria monocytogenes in the different cheeses that were produced are illustrated in FIG. 7. The results are significantly different between the population levels in the control tests in the presence of the commercial fermenting agent Holdbac® and in the presence of the 2 protective cultures of Carnobacterium maltaromaticum. The differences between the population levels appeared after 5 days of production and became more apparent during ripening. The population of Listeria monocytogenes stabilized after 15 days of ripening in the presence of the protective culture Carnobacterium maltaromaticum F88, while it continued to increase in the presence of Carnobacterium maltaromaticum B10 or the commercial culture Holdbac®. In contrast, strains F88 and B10 exhibited a better inhibiting activity for Listeria monocytogenes compared with the commercial protective culture Holdbac®.

### Example 5: Production of Semi-Soft Saint Nectaire Cheese

### Protocol for Production of Semi-Soft Saint Nectaire Cheese

Production corresponded to 10 vats of 5 L of milk (1 cheese of 450 g/vat) inoculated with the mixture of Listeria monocytogenes strains. From these, two series of three vats were inoculated with the protective cultures Carnobacterium maltaromaticum CNCM I-5242 (in the experiments below, represented by F88) and Carnobacterium maltaromaticum CNCM I-5243 (in the experiments below, represented by B10) and two vats with the commercial protective culture Holdbac®. Two vats without a protective culture acted as controls for the growth of Listeria monocytogenes.

The milk used was pasteurized and standardized milk (MF/PC=1). Inoculation of the fermenting agents was carried out to a level of 1% in the milk before coagulation and a mixture of bacteria for ripening was sprayed onto the surface on D5 and D9.

The Carnobacterium maltaromaticum protective cultures were frozen ready-to-use cultures. Inoculation was similar to that carried out for the soft cheese technique, namely 50 mL in 5 L of milk before coagulation. The mixture of strains and the inoculation conditions for Listeria monocytogenes were similar to those used for the production of soft cheese.

Ripening was carried out for 28 days.

Sampling timing and purpose:
T0 milk before coagulation=inoculation of strains+Listeria=30 mL of milk removed and frozen at −20° C.
T1 d=¼ cheese 25 g for counting of Listeria monocytogenes.
T8 d=¼ cheese: 25 g for counting of Listeria monocytogenes
T18 d=¼ cheese: 25 g for counting of Listeria monocytogenes
T28 d=¼ cheese: 25 g for counting of Listeria monocytogenes.

The Listeria monocytogenes were counted on a mixture of curd and crust.

The pH of the samples that were taken was also measured.

### Result:

The appearance of all of the cheeses produced during the experiment was correct throughout production. All of the cheeses had the same appearance.

The pH of the cheeses produced and the dry extracts obtained were correct throughout production.

The population numbers for Listeria monocytogenes in the different cheeses that were produced are illustrated in FIG. 8.

The results for the production of Saint Nectaire type cheese are similar to those obtained for the soft cheese technique:
the population of Listeria monocytogenes was lower than that of the controls in the presence of 2 test protective cultures of Carnobacterium maltaromaticum;
the efficacy of the 2 protective cultures of Carnobacterium maltaromaticum was higher than that of the commercial protective culture
the protective culture Carnobacterium maltaromaticum F88 exhibited the best inhibition results.

1.-11. (canceled)

12. An isolated strain of Carnobacterium maltaromaticum selected from Carnobacterium maltaromaticum, deposited under the number CNCM I-5242 and Carnobacterium maltaromaticum, deposited under the number CNCM I-5243.

13. A bacterial preparation comprising the strain CNCM I-5242 or the strain CNCM I-5243.

14. The bacterial preparation as claimed in claim 13, said preparation further comprising a lactic fermenting agent and/or a ripening fermenting agent.

15. The bacterial preparation as claimed in claim 13, wherein said preparation is a concentrate, a frozen concentrate, or a lyophilizate.

16. The bacterial preparation as claimed in claim 13, wherein said preparation is a protective culture of Carnobacterium maltaromaticum.

17. A method of preparing a food product, comprising adding the isolated strain as claimed in claim 12 or a bacterial preparation comprising said isolated strain to the food product.

18. The method as claimed in claim 17, wherein said isolated strain or said bacterial preparation inhibits the growth of bacteria of the genus Listeria in said food product.

19. The method as claimed in claim 17, wherein said food product is a fermented milk product, in particular cheeses, fermented milk.

20. A method for the preparation of a cheese, said method comprising:
inoculating at least one isolated strain of Carnobacterium maltaromaticum as claimed in claim 12 or a bacterial preparation comprising said at least one isolated strain, into milk or during the transformation of milk.

21. The method for the preparation of a cheese as claimed in claim 20, wherein inoculating with the Carnobacterium maltaromaticum bacteria is from 10² CFU·mL⁻¹ to 10¹¹ CFU·mL⁻¹, in particular from 10⁶ to 10⁷ CFU·mL⁻¹, with respect to the volume of milk.

22. A method for reducing microbial contamination during a process for the production of a fermented milk product, comprising adding a quantity of 10² CFU·mL⁻¹ to 10¹¹ CFU·mL⁻¹, in particular from 10⁶ to 10⁷ CFU·mL⁻¹, with respect to the volume of the milk product, of the isolated strain of Carnobacterium maltaromaticum as claimed in claim 12 or a bacterial preparation comprising said isolated strain.
